(19) United States
(12) Patent Application Publication
(10) Pub. No.: US 2025/0235564 A1
(43) Pub. Date: Jul. 24, 2025

(54) COMPOSITE INK FORMULATIONS FOR ENDOSCOPIC IMAGING

(71) Applicants: ARIZONA BOARD OF REGENTS ON BEHALF OF ARIZONA STATE UNIVERSITY, Scottsdale, AZ (US); MAYO FOUNDATION FOR MEDICAL EDUCATION & RESEARCH, Rochester, MN (US)

(72) Inventors: Subhadeep DUTTA, Tempe, AZ (US); Jordan YARON, Scottsdale, AZ (US); Rahul PANNALA, Scottsdale, AZ (US); Kaushal REGE, Chandler, AZ (US); Mallikarjun GOSANGI, Tempe, AZ (US)

(21) Appl. No.: 18/700,664

(22) PCT Filed: Oct. 11, 2022
(86) PCT No.: PCT/US2022/046338

§ 371 Date: Apr. 11, 2024

Related U.S. Application Data
(60) Provisional application No. 63/254,523, filed on Oct. 11, 2021.

(51) Int. Cl. A61K 49/00; A61B 90/00

(52) U.S. Cl. CPC A61K 49/006; A61B 90/39; A61K 49/0093; A61B 2090/3908; A61B 2090/3954

(57) ABSTRACT

The present invention biomaterial-based composite ink formulations for use in endoscopic imaging (including in colon tattooing) procedures and their methods of production. The biomaterial-based composite ink formulations comprise at least one contrast agent selected from the group consisting of: iron oxide, copper oxide, carbon nanotube, and graphene oxide; and at least one polysaccharide-based biomaterial selected from chitosan or its derivative. The contrast agents are preferably nanoparticles.

### CROSS-REFERENCE TO RELATED APPLICATIONS

This application claims the benefit of U.S. Provisional Application No. 63/254,523, entitled “Composite Ink Formulations for Endoscopic Imaging,” which was filed Oct. 11, 2021, the entire disclosure of which is hereby incorporated herein by this reference.

### STATEMENT REGARDING FEDERALLY SPONSORED RESEARCH OR DEVELOPMENT

This invention was made with government support under R01 EB020690 awarded by the National Institutes of Health. The government has certain rights in the invention.

### FIELD OF THE INVENTION

The invention relates to compositions for endoscopic tattooing and methods of producing thereof.

### BACKGROUND OF THE INVENTION

Endoscopic tattooing (or commonly known as “colon tattooing”) is a clinical technique often used along with endoscopic imaging procedures to mark lesions or polyps in the colon for surgical resection or to guide follow-up procedures (general guidelines are 3 cm distal to the lesion at total three sites). For example, colon tattooing procedures are usually recommended for lesions with evident/suspected colorectal cancers or with lesions with ≥2 cm that have a potential risk of cancer and for flat broad lesions. Lesions can be removed by endoscopic submucosal dissection (ESD) or endoscopic mucosal resection (EMR) procedures. Currently the widely used commercial ink for clinical colon tattooing procedure is the carbon black-based Spot-Ex® (GI Supply) endoscopic marker. This ink comprises carbon black particles stabilized in a water/glycerin (4:1 v/v) mixture. Spot-Ex® and the related and popularly used India ink possess several adverse effects (for example, inflammation, peritonitis, fibrosis) and poor spot localization due to diffusion throughout the submucosal tissue over time. Recent evidence suggests that currently used colon inks also diffuse beyond the submucosa to distal locations within tissues and may be associated with further adverse effects. This rapid and uncontrolled diffusion of black tattoo ink throughout the submucosal tissue, further complicates the purpose of its intended use. Thus, improvements in contrast agents for endoscopies are needed.

### SUMMARY OF THE INVENTION

Described herein are a series of new biomaterial formulations containing contrast agents that address clinical complications from Spot-Ex® and India ink. The described compositions possess enhanced mucoadhesivity to help retain localization in submucosal injection sites by interactions with connective tissue matrices, high contrast ratio visualization by using composite ink formulations with high optical density, and carrier materials which are well established to have high biocompatibility and controllable degradability.

The disclosed compositions demonstrate high contrast towards endoscopic imaging and ability to retain the localized tattoo mark due to its inherent mucoadhesivity, and as consequence very low diffusion/transport compared to clinically used Spot-Ex® ink.

Novel biomaterial-based inks were formulated in combination with (1) metallic nanoparticles (such as dextran-coated negatively-charged iron-oxide nanoparticles) as contrast agents and (2) mucoadhesive cationic biomaterials (such as chitosan-derived polymers) as a protective coating with high spot-retention capability, for endoscopic imaging applications in the GI tract. These formulations represent a new class of biocompatible endoscopic tattoo inks, which improve the quality of endoscopic procedures with more-accurate, safe marking for long-term visualization and offer possibilities of non-invasive follow-up procedures in conjunction with MR or CT imaging.

In some aspects, the disclosure concerns compositions of biomaterial-based composite ink comprising at least one contrast agent selected from the group consisting of iron oxide, copper oxide, carbon nanotube, and graphene oxide; and at least one polysaccharide-based biomaterial selected from chitosan or its derivative. Some inks comprise iron oxide.

Other aspects of the disclosure concern methods of endoscopic tattooing comprising injecting to tissue a composition comprising biomaterial-based composite ink comprising at least one contrast agent selected from the group consisting of iron oxide, copper oxide, carbon nanotube, and graphene oxide; and at least one polysaccharide-based biomaterial selected from chitosan or its derivative.

Further aspects of the disclosure concern methods of synthesizing a composition of biomaterial-based composite ink comprising mixing: at least one contrast agent selected from the group consisting of iron oxide, copper oxide, carbon nanotube, and graphene oxide; and at least one polysaccharide-based biomaterial selected from chitosan or its derivative.

Nanoparticles of any suitable size may be utilized. In some embodiments, the nanoparticles are deliverable through an endoscopic injection needle as a liquid ink.

In certain embodiments, the at least one contrast agent is coated. One suitable coating for the at least one contrast agent is dextran. In some embodiments, the at least one contrast agent is a nanoparticle. In some embodiments, the contrast agent is ‘Dextran-coated Iron Oxide nanoparticles’ (which is the ‘parental formulation’ without chitosan coating) and, in certain embodiments, its hydrodynamic diameter is broadly ˜250 nm or so as determined by Dynamic Light Scattering (DLS). In some embodiments, these nanoparticles are ˜10 nm in diameter and clustered together due to their strong magnetic property/attraction. Some chitosan-based-Iron Oxide formulations have size ranging broadly from 1-5 microns.

Some polysaccharide-based biomaterial is selected from the group consisting of: cysteine-modified chitosan, glutathione-modified chitosan, and catechol-modified chitosan. Certain composite inks comprise dextran-coated iron oxide nanoparticles or dextrose coated iron nanoparticles encapsulated with (a) quaternized Chitosan, (b) medium molecular weight Chitosan, or (c) high molecular weight Chitosan.

### BRIEF DESCRIPTION OF THE DRAWINGS

FIG. 1 presents schematics of action of the novel biomaterial-based Endoscopic tattooing ink; developed by encapsulating metallic iron oxide (IO) nanoparticles (used as contrast agent) with chitosan-derived biopolymers (used as retention-mediating carrier).

FIG. 2 depicts, in accordance with certain embodiments, representative images of the ex vivo performance of disclosed composite inks.

FIG. 3 depicts, in accordance with certain embodiments, exemplary in vivo results of the disclosed composite inks.

FIG. 4 illustrates, in accordance with certain embodiments, synthesis of quaternized-chitosan.

FIG. 5 presents, in accordance with certain embodiments, a scheme for synthesis of GSH-conjugated chitosan.

FIG. 6 presents, in accordance with certain embodiments, schematics of stepwise synthesis of dextran-coated Iron Oxide (IO) nanoparticles (as parental tattoo-ink formulation) by co-precipitation reaction, followed by encapsulation reaction to prepare chitosan-based composite tattoo-ink formulations.

FIG. 7 presents, in accordance with certain embodiments, characterization of dextran-coated iron oxide nanoparticle (DFeNP).

FIG. 8 presents, in accordance with certain embodiments, characterization of dextran-coated iron oxide nanoparticle (DFeNP).

FIGS. 9A-9D show, in accordance with certain embodiments, scanning electron microscopy (SEM) images of dextran-coated Iron Oxide (IO) nanoparticles (FIG. 9A), high molecular weight-chitosan coated IO-nanoparticles (hC-IO) (FIG. 9B), glutathione-conjugated chitosan coated IO-nanoparticles (gC-IO) (FIG. 9C), and quaternized-chitosan coated IO-nanoparticles (qC-IO) (FIG. 9D). Scale bars are 200 nm.

FIG. 10A shows a comparison of free amine present in different chitosan derivatives measured with Ninhydrin assay. FIGS. 10B and 10C depict an exemplary in vitro inflammation assay response of different tattoo-ink components such as IO nanoparticles and chitosan derivatives towards IRF3 (FIG. 10B) and NFKB activation (FIG. 10C) with J774-dual mouse-derived macrophage-based cells. ‘Control’ refers to culture media as negative control and ‘LPS’ refers to positive control. FIG. 10D depicts representative images of gel clot assay to detect endotoxin present. (I) represents positive (P) and negative (N) controls of the assay with detection limit of 0.25 EU/mL. (II) represents no gel clot formation in different neutralized chitosan derivatives used (marked as 1-4) and different batches of synthesized 10 nanoparticles (marked as A, B). (III) represents no gel clot formation in chitosan-based IO-composite tattoo-inks (marked as 1B-4B)

FIG. 11A presents physical appearance of different tattoo-inks taken in a cuvette and a table depicting hydrodynamic diameter & zeta potential measurements of different tattoo-inks using dynamic light scattering, FIG. 11B depicts representative images of ex vivo tattoo performed with different tattoo-ink formulations using fresh porcine intestinal tissue. FIG. 11C presents exemplary images of preliminary trials (N=1) of endoscopic injections into esophagus and stomach of a just-sacrificed porcine with the commercial Spot® Ex ink and synthesized IO-nanoparticles.

FIG. 12 shows that at two days shows the injections with Spot® Ex in merged into one bigger spot while two injections using DFENP remained as two spots.

FIG. 13 shows, in accordance with certain embodiments, representative images at 28 days for an in vivo first cohort mice sturdy using Spot® Ex, LCH-DFeNP, HCH-DFeNP and DFeNP.

FIG. 14 presents representative 28-days for an in vivo second cohort mice study using Spot-EX, MCH-DFeNP, QCH-DFeNP, and GCH-DFeNP.

FIG. 15 presents graphs showing size of the spot area at 28 days.

FIG. 16 presents graphs showing the contrast of the dye spot at 28 days.

FIG. 17 shows photos and H&E of the dye injection sites.

FIG. 18 presents illustrations and hematoxylin-eosin stain (H&E) of the dye injection sites are shown in FIG. 17. Hyperkeratosis scores, cellularity scores, and fibrotic capsule scores for the tested dyes.

FIG. 19A shows schematics of randomized subcutaneous tattoo-ink injections during 28-day in vivo studies in live Balb/c mice. FIG. 19B presents exemplary images of tattoo-ink implanted mice at the start (day-0) and end (day-28) of the in vivo studies. FIGS. 19C and 19D compares the spot-area (FIG. 19C) and contrast quantification (FIG. 19D) between commercial ink (Spot® Ex) and our tattoo-ink formulations. FIG. 19E displays representative gross pathology and H&E-stained images of skin tissues isolated following sacrificing the mice at end of the study (day-28).

It is understood that the embodiments of the invention herein described are merely illustrative of the application of the principles of the invention. Reference herein to details of the illustrated embodiments is not intended to limit the scope of the claims.

### DETAILED DESCRIPTION OF THE INVENTION

Detailed aspects and applications of the invention are described below in the drawings and detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the invention. It will be understood, however, by those skilled in the relevant arts, that the present invention may be practiced without these specific details. It should be noted that there are many different and alternative configurations, devices, and technologies to which the disclosed inventions may be applied. The full scope of the inventions is not limited to the examples that are described below.

Endoscopic tattooing (including “colon tattooing”) is a routine clinical technique to mark colorectal and other gastrointestinal lesions (i.e., polyps in the colon) for successive surgical resection or for facilitating follow-up of the endoscopic resection site. The technique involves injecting a dark-colored (high contrast) ink into the submucosal layer of the colon. Currently used inks, such as Spot-Ex® (GI Supply) or India ink, are prone to several side effects (Acta Med Port. 2012, 25 (5), 288) including abscess formation, peritonitis and/or development of inflammatory bowel disease (Gastrointestinal Endoscopy 1999, 49 (5), 636). Additionally, submucosal fibrosis associated with existing inks can interfere with endoscopic resection techniques later and may increase the risk for incomplete resection and/or perforation. Lastly, Spot-Ex® and related dyes diffuse throughout the submucosal tissue, complicating juxtaposition of the tattoo with the intended lesion of interest.

There is a significant, unmet clinical need for an alternative contrast agent/ink to safely mark the lesion for endoscopic tattooing procedure and which can retain localization by minimizing transport in and across tissues for improved follow-up accuracy and does not induce toxic, inflammatory, or fibrotic responses. Described herein are biomaterial-based composite ink formulations for use during endoscopic imaging procedures to mark any tissue along the gastrointestinal tract, including the colon, esophagus, pancreas, and stomach. In some aspects, the biomaterial-based composite ink formulations is used for colon tattooing. The biomaterial-based composite ink compositions comprise at least one contrast agent selected from the group consisting of: iron oxide, copper oxide, carbon nanotube, and graphene oxide; and at least one polysaccharide-based biomaterials. The polysaccharide-based biomaterials function as coating materials around the contrast nanoparticles to impart a mucoadhesive property, which improves retention and localization at the site of injection in the submucosa.

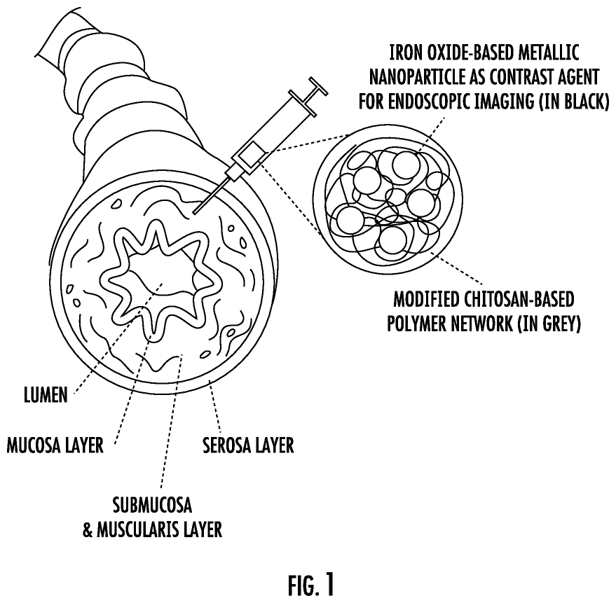

FIG. 1 presents schematics of injecting the novel biomaterial-based endoscopic tattooing ink; developed by encapsulating metallic nanoparticles (used as contrast agent) with chitosan-derived biopolymers (used as retention-mediating carrier).

The contrast agents may be nanoparticles. The polysaccharide-based biomaterials include dextran, chitosan, or their derivatives. Iron oxide, copper oxide, carbon nanotube, and graphene oxide are selected as contrast agents because of their inherent dark grey/black color. To synthesize the disclosed biomaterial-based composite ink, the polysaccharide-based biomaterials are vigorously mixed with the contrast agent nanoparticles, for example, for 24 hours. The ratio between contrast agent and the biomaterial coating can be modulated for optimization.

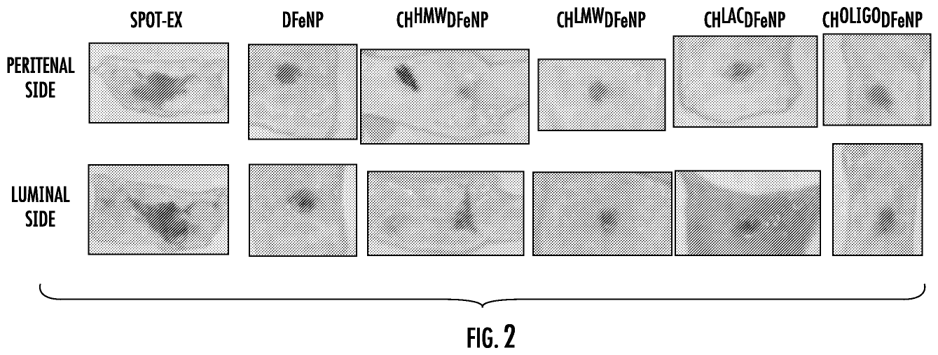

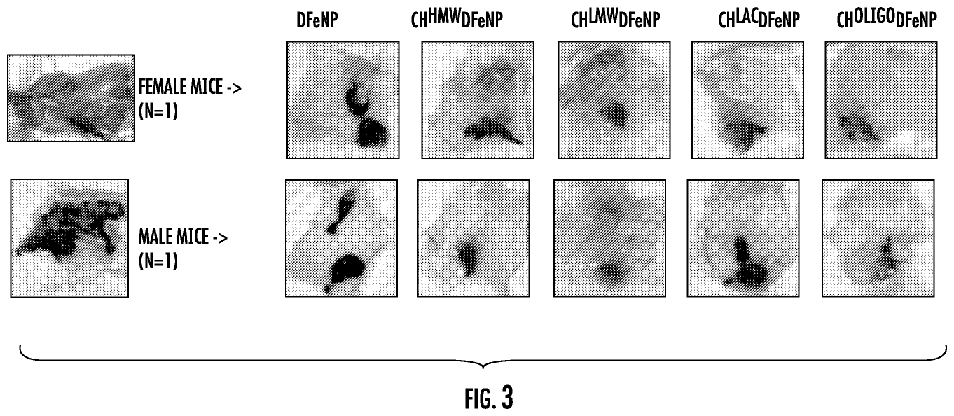

The disclosed biomaterial-based composite ink possesses strong contrast due to dark brown/black color; can retain localization at injection site for improved follow-up accuracy; and induce low cytotoxic effects and low inflammatory response (see FIG. 3). Furthermore, the tattoo mark formed from the disclosed biomaterial-based composite ink can be assessed with MRI and X-ray CT in addition to endoscopic imaging under regular white light. As shown in the examples, the disclosed composite inks demonstrate near-equivalent contrast quantification to commercially available inks while also exhibiting substantially improved spot localization (FIG. 2).

Also described herein are method of producing the biomaterial-based composite ink formulation.

### Polysaccharide-Based Biomaterials-Chitosan Modifications

Chitosan (an amine-rich positively charged natural biopolymer) is well-known in literature for its mucoadhesive property due to its electrostatic interaction with mucin. Chitosan can be categorized into different sub-categories based on its molecular weight (e.g., high, medium, low), degree of deacetylation (DDA, varying as 40%, 60%, 70%, 80%, 90%), terminal functional group (e.g., chitosan-lactate (CH-Lactate), chitosan-oligo (CH-oligo), carboxymethyl-chitosan, quaternized chitosan (Q-CH), and thiolated chitosan). The functional moieties in chitosan play a role in determining toxicity, mucoadhesivity, and the degree of inflammatory response induced. The addition of thiol and catechol groups increases the mucoadhesivity due to interaction with thiols and amines present in mucin, respectively.

In some embodiments, the molecular weight (MW) ranges of chitosan are as follows: high MW being 310-375 kDa, medium MW being 190-310 kDa, low MW being 50-190 kDa. Depending on formulation, any one or mixture of the high, medium, or low molecular weight chitosan may be utilized.

Chitosans with different degrees of deacetylation were evaluated. In some embodiments the degree of deacetylation than 75% or greater. In principle lower the degree of deacetylation indicates lower the % of free primary amine present in chitosan, which makes the chitosan biopolymer less cationic and may indicate that it would bind less strongly with negatively charged mucin in the submucosal layer. However, any degree of acylation that provides suitable performance (for example, with regard to mucin binding the submucosal layer) is acceptable.

CH-Oligo of any suitable molecular weight may be utilized. In some embodiments, a molecular weight near or less 1000 Da was utilized.

In some embodiments, the composition comprises a high molecular weight (310-375 kDa) chitosan (CH) modified with EDC-NHS catalyzed coupling reaction (at pH: 5-6) with cysteine, glutathione (thiol-containing tripeptide), or hydrocaffeic acid. In some aspects, the polysaccharide-based biomaterials in the composition is cysteine-modified chitosan (CH-Cys), glutathione-modified chitosan (CH-GSH), or catechol-modified chitosan (CH-Cat). In other embodiments, chitosan treated with Trout's reagent, which is a class of thiolated-chitosan without consuming its amine groups, is used in the biomaterial-based composite ink.

### Contrast Agents

Preferably, the contrast agent nanoparticles have a partial negative surface charge, which facilitates its binding with the chitosan derivatives, which are polycationic, in the biomaterial-based composite ink. Thus, in some aspects, the contrast agent nanoparticles are coated, for example, with dextran or other coating to provide a partial negative surface charge. In such embodiments, the contrast agents are iron oxide or copper oxide. In other aspects, negatively charged carbon nanotubes and graphene oxide nanoparticles can be used as un-coated contrast agents.

Concerning nomenclature used herein, “DFe” refers to dextran coated iron particles. CH—YYY or CHYYY refer to chitosan modified with a particular compound (designated “YYY” as a generic substitute for the actual compound). DFe CHYYY refers to a complex between dextran coated iron particles and chitosan modified with YYY.

### EXAMPLES

The present invention is further illustrated by the following examples that should not be construed as limiting. The contents of all references, patents, and published patent applications cited throughout this application, as well as the Figures, are incorporated herein by reference in their entirety for all purposes.

All the chemicals were purchased from Sigma-Aldrich unless specified otherwise and were used as received. High (2% (w/v)) and low (4% (w/v)) molecular weight Chitosan solutions were purchased from Tidal Vision. Dextran sulphate (MW-40,000) sodium salt was purchased from ICN Chemicals. Ammonium hydroxide (25% (v/v)) solution and Miracloth were purchased from EMD Millipore. Dialysis bags (with molecular weight cut-off (MWCO)-3500 Da) was purchased from spectrapor. Clinically used Spot® Ex endoscopic tattoo ink (GIS-45) by GI Supply were supplied generously from Mayo Clinic, Arizona. Fresh porcine intestines were collected from a local meat processing plant named West Valley processing. MilliQ water (18.2 M (2·cm) was used as solvent for all experiments conducted, unless mentioned otherwise.

Primarily three different types of commercial chitosan were used in this study-high molecular weight or ‘hC’ (310-375 kDa), medium molecular weight or ‘mC’ (190-310 kDa), low molecular weight or ‘IC’ (50-190 kDa).

### Example 1. Chitosan Modifications

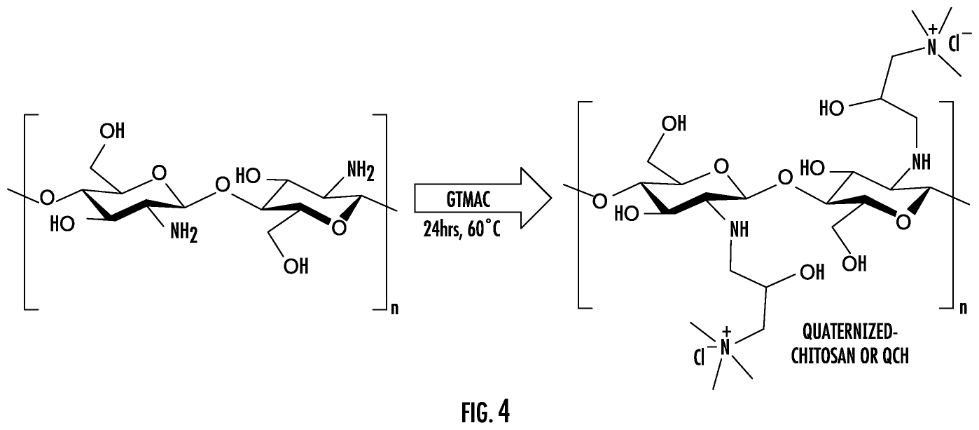

Glutathione-conjugated Chitosan (gC) synthesis: High molecular weight chitosan (2% (w/v)) solution were adjusted to pH ˜6.5 with dropwise addition of 1M sodium hydroxide (NaOH) solution and further diluted with water to make a final concentration of 0.7 wt %. Approximately 880 mg of L-glutathione reduced (thiol containing tri-peptide), 270 mg of 1-ethyl-3-(3-dimethylaminopropyl) carbodiimide (EDC) and 160 mg of N-Hydroxysuccinimide (NHS) were added in 10 mL of water, taken in a 50 mL falcon tube and vortexed well to dissolve. This solution mixture was dropwise added into 30 mL of chitosan solution (prepared before) under vigorous stirring at a speed of 700 rpm. Final pH of the reaction was adjusted between 5-6. This reaction mixture was stirred for 48 hours to complete the conjugation reaction (schematics shown in FIG. 4) and further dialyzed against Milli-Q water (pH˜5) for 48 hours at 4° C. using a 3.5 kDa molecular weight cutoff (MWCO) dialysis membrane. Thereafter purified glutathione-conjugated chitosan (gC) polymer product was collected with freeze drying (LABCONCO Freezone 4.5 L freeze-dryer at −84° C. with 0.013 mbar pressure for 2 days) the dialyzed solution.

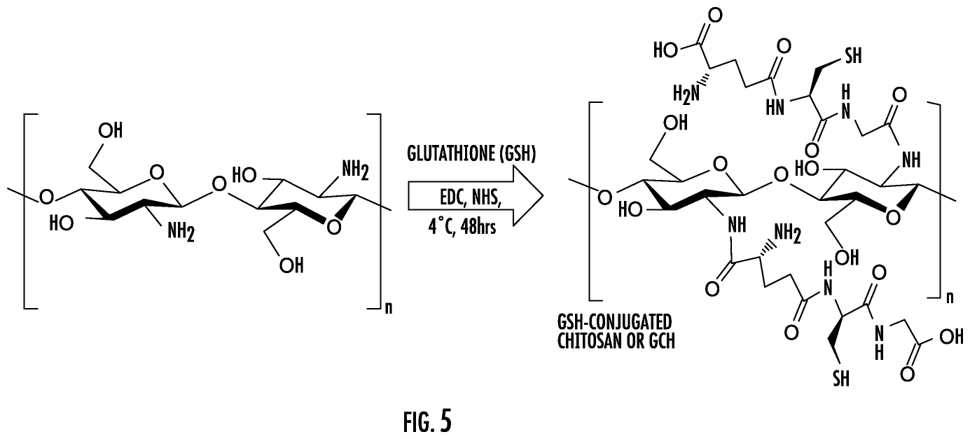

Quaternized Chitosan (qC) synthesis: High molecular weight chitosan (2% (w/v)) solution were adjusted to pH ˜6.5 with dropwise addition of 1M sodium hydroxide (NaOH) solution and further diluted with water to make a final concentration of 1 wt %. 15 mL of 1% (w/v) chitosan solution was taken in a 20 mL scintillation vial and a total of 750 μL of glycidyltrimethylammonium chloride (GTMAC) was added into it in three equal portions at every 2.5 hours interval. Afterwards the final reaction mixture was allowed to stir at 65° C. at a speed of 400 rpm for 24 hours (schematics shown in FIG. 5) and further dialyzed against Milli-Q water for 4 days at 4° C. using a 3.5 kDa molecular weight cutoff (MWCO) dialysis membrane. Thereafter purified quaternized chitosan (qC) polymer product was collected with freeze drying (LABCONCO Freezone 4.5 L freeze-dryer at −84° C. with 0.013 mbar pressure for 2 days) the dialyzed solution.

### Example 2. Chitosan Modifications

Chitosan (amine-rich positively charged natural biopolymer) is well-known in literature for its mucoadhesive property due to its electrostatic interaction with mucin. Chitosan can be categorized into different sub-categories based on its molecular weight (e.g., high (HWM-CH), medium (MMW-CH), low (LMW-CH)), degree of deacetylation (DDA, varying as 40%, 60%, 70%, 80% approximately), terminal functional group (e.g., chitosan-lactate (CH-Lactate), chitosan-oligo (CH-oligo), carboxymethyl-chitosan, quaternized chitosan (Q-CH), and thiolated chitosan). The functional moieties in chitosan play a role in determining toxicity, mucoadhesivity, and degree of inflammation response. With the addition of thiol and catechol groups increases the mucoadhesivity due to interaction with thiols and amines present in mucin, respectively. Herein we have modified high molecular weight (310-375 kDa) chitosan (CH) with 1-Ethyl-3-(3-(dimethylamino)-propyl) carbodiimide hydrochloride (EDC) and N-hydroxysuccinamide (HHS) catalyzed coupling reaction (at pH: 5-6) with cysteine (CH-CYS), glutathione (thiol-containing tripeptide) and hydrocaffeic acid and successfully synthesized cysteine-modified chitosan (CH-Cys), glutathione-modified chitosan (CH-GSH), catechol-modified chitosan (CH-Cat) respectively. In addition, by treating with Trout's reagent, another class of thiolated-chitosan without consuming its amine groups was synthesized. All modified materials were characterized using FTIR spectroscopy, and amine, thiol, and catechol concentration after conjugation was measured using Ninhydrin assay, Elman's assays, and UV-based assay respectively. Quaternized chitosan were also generated for these purposes.

### Example 3. Synthesis of Contrast Nanoparticles

Schemes for synthesis of modified chitosan (quaternized-chitosan and gluutathione (GSH)-conjugated chitosan) are shown in FIGS. 4 and 5. For quaternized-chitosan, chitosan was treated with glycidyltrimethylammonium chloride (GTMAC) at 60° C. for 24 hours. For GSH-conjugated chitosan, chitosan was treated with GSH, 1-Ethyl-3-(3-(dimethylamino)-propyl) carbodiimide hydrochloride (EDC) and N-hydroxysuccinamide (HHS) for 48 hours at 4° C.

Ninhydrin Assay: Reactive primary amine content of different chitosan derivatives was determined using ninhydrin assay. Briefly, 190 μL of glacial acetic acid solution (0.05% v/v acetic acid in water) and adequate volumes of neutralized chitosan solutions were mixed in 1.5 mL Eppendorf tubes. Ninhydrin reagent (100 μL) was added to all samples, which were mixed thoroughly using a vortex shaker. The mixtures were immediately placed in a 100° C. water bath for 10 minutes and allowed to cool down to room temperature. Thereafter 500 μL of 95% ethanol was added to stabilize the generated complex between ninhydrin and reactive primary amines present in chitosan. Absorbance values of the samples at 570 nm wavelength were then determined using a BioTek Synergy 2 plate reader. Amine content of different chitosan derivatives were quantified using a calibration curve obtained with glycine standards.

Elman's Assay: Free thiol groups immobilized on glutathione-conjugated chitosan (gC) were estimated spectrophotometrically using Ellman's reagent. Briefly 250 μL of reaction buffer (which contains 100 mM sodium phosphate and 1 mM EDTA at pH-8), 5 μL of Ellman's reagent (which was prepared by dissolving 80 mg of Ellman's reagent in 20 mL of reaction buffer), and 25 μL of neutralized glutathione-conjugated chitosan solution were mixed in a 1.5 mL Eppendorf tube and incubated at room temperature for 15 minutes. Then absorbance at 412 nm was measured using a BioTek Synergy 2 plate reader. Thiol content of glutathione-conjugated chitosan (gC) samples were then quantified using a calibration curve obtained with cysteine standards.

### Example 4. Preparation of Dextran-Coated Iron-Oxide Nanoparticles (IO)

Dextran-coated iron oxide nanoparticles were synthesized using a one-pot co-precipitation method in the presence of dextran sulphate, ferrous sulphate & ferric chloride as precursor (FeSO₄: FeCl₃-1:2 mole ratio). Briefly, 250 mg of dextran sulfate was dissolved in 15 mL water. Afterwards 450 mg iron (III) chloride (FeCl₃) and 320 mg of iron (II) chloride tetrahydrate (FeSO₄·4H₂O) were dissolved in 15 mL water with vortexing, taken in a 50 mL falcon tube by maintaining a 1:2 mole ratio of FeSO₄: FeCl₃ and the resultant solution was poured into the previous solution containing dextran sulfate; allowed to mix thoroughly for 15 mins. Meanwhile 7% (v/v) ammonium hydroxide (NH₄OH) solution was prepared by appropriately diluting 25% (v/v) ammonium hydroxide solution with water. Then ˜40 mL of 7% (v/v) ammonium hydroxide (NH₄OH) solution was dropwise added to the precursor solution (containing dextran sulphate, ferrous sulphate & ferric chloride) with vigorously stirring at 700 rpm to bring the pH of the solution between 10-11. The entire solution mixture was then heated at 65° C. for 30 mins to complete the co-precipitation reaction and followed by filtration with miracloth to remove the larger aggregates. Subsequently the filtrate solution mixture was centrifuged at a speed of 6000 g at 4° C. and further washed three times with water to remove the excess amount of precursor dextran sulphate and ammonium hydroxide. Finally, the purified dextran coated iron oxide nanoparticles (IO) were dispersed in 0.1×PBS buffer (pH˜7.4) at a stock concentration of 25 mg/mL and stored under dark in 4° C. for future use.

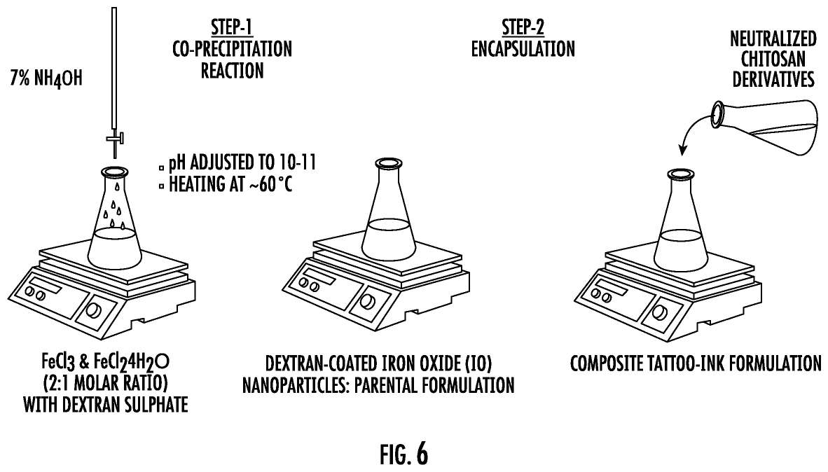

FIG. 6 presents schematics of stepwise synthesis of dextran-coated Iron Oxide (IO) nanoparticles (as parental tattoo-ink formulation) by co-precipitation reaction, followed by encapsulation reaction to prepare chitosan-based composite tattoo-ink formulations.

### Example 5. Chitosan-IO Composite Tattoo-Ink Preparation & Characterization

Equal volumes of IO-nanoparticle stock solution (25 mg/mL) and different kinds of neutralized chitosan solutions (hC, mC, IC, gC, qC) were thoroughly mixed by vortexing overnight to encapsulate the IO-nanoparticles with chitosan-derived biopolymers. This has formulated five kinds of composite tattoo-ink solutions namely high molecular weight chitosan-coated IO-nanoparticles (hC-IO), medium molecular weight chitosan-coated IO-nanoparticles (mC-IO), low molecular weight chitosan-coated IO-nanoparticles (IC-IO), glutathione-conjugated chitosan-coated IO-nanoparticles (gC-IO), quaternized chitosan-coated IO-nanoparticles (qC-IO).

Dynamic light scattering was performed by following the above-mentioned procedure to measure the hydrodynamic diameter and zeta potential of these composite tattoo-ink formulations. SEM images were captured following the same above-mentioned procedure to visualize the composite tattoo-ink microparticles.

### Example 6. Characterization of Dextran-Coated Iron-Oxide Nanoparticles (IO)

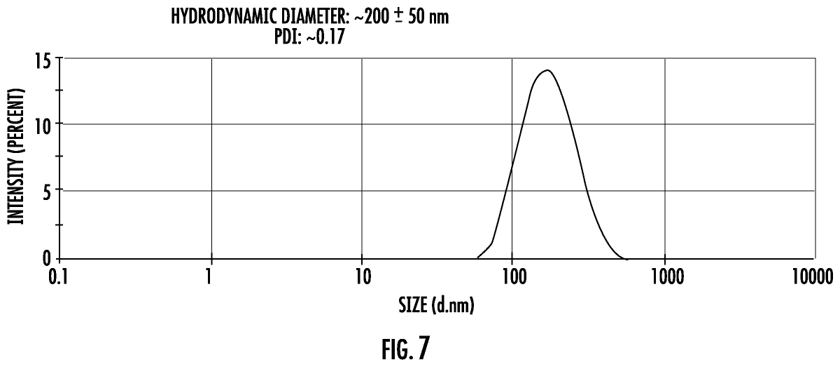

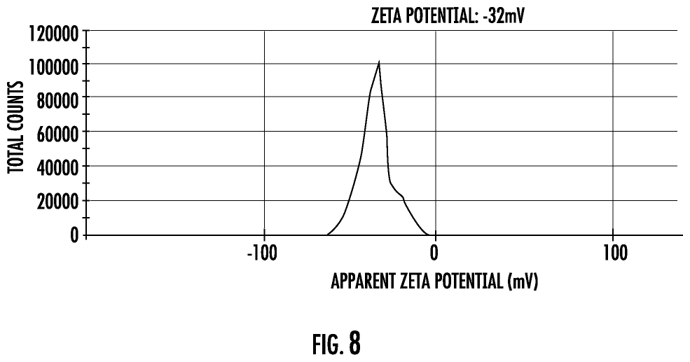

Dynamic Light Scattering: Hydrodynamic diameter and surface zeta potential of dextran coated iron oxide nanoparticles (IO) were determined with the principle of dynamic light scattering (DLS) using a Zetasizer Nano-ZS instrument by Malvern. Plots of this information are presented in FIGS. 7 and 8. Anoparticle stock solutions were diluted in 1:10 ratio with 1×PBS buffer (pH˜7.4) prior to DLS measurements to determine hydrodynamic diameter and surface zeta potential.

Electron Microscopy: Firstly, IO nanoparticle stock solutions were diluted in 1:50 ratio with 0.1×PBS buffer (pH˜7.4) and sonicated inside ice bath for 6 hours to make well-dispersed homogeneous solution. This solution was further drop-casted onto TEM grids and samples were air-dried overnight. Nanoparticles were visualized using a Philips CM200-FEG high resolution transmission electron microscopy (HRTEM) instrument (operating at an accelerating voltage 200 kV) in the LeRoy Eyring Center for Solid State Sciences at ASU. Briefly, the perimeter of ˜25 nanoparticles was individually measured using ImageJ software (U.S. National Institutes of Health, Bethesda, MD, USA; https://imagej.nih.gov/ij/; 1997-2018) and diameter of the nanoparticles were calculated accordingly. Elemental analysis on the same TEM grids were carried out using a JEOL ARM200F STEM instrument to determine the composition of the nanoparticles formed. Elemental analysis acquired from EDS was conducted under atomic resolution mapping followed by visualized indexing.

The surface morphology of IO nanoparticles was visualized using a Zeiss Auriga focused ion beam scanning electron microscopy (FIB-SEM) at 25 kV. Nanoparticles droplet was placed on a double-sided carbon adhesive tape attached on the aluminum stub and were sputter-coated with carbon layer for 120 s using a Denton TSC carbon sputter coater instrument, prior to SEM visualization.

Inductively Coupled Plasma-Optical Emission Spectrometry (ICP-OES); ICP-OES was used to accurately quantify the iron (Fe) content present in IO nanoparticles. Known volume of IO nanoparticle stock solution was digested in aqua regia (1:3 mixture of ultrapure HNO3 and HCl by volume) at 95° C. for 4 h and then it was evaporated to obtain oxidized solid residue. The digested residues were then serially diluted with 2% (v/v) nitric acid (HNO3) to bring the iron concentration within detectable range and passed through 0.45 μm syringe filter. The concentration of iron was then measured using an Agilent 5900 SVDV ICP-OES instrument, with a detection limit of low to sub-ppb (ug/L) level. VHG-SM75B-500 solution (with known Fe-content) was used as a standard to obtain a calibration curve and then CCV standard solution (product no. 4400-010100 from CPI International) was used as an internal calibration. Two different wavelengths of detection (234.35 nm, 238.204 nm) were used to measure iron content using this ICP-OES instrument.

Raman Spectroscopy: IO nanoparticles were freeze-dried to obtain solid powders for evaluating with Raman spectroscopy. Oxide-coated silicon (Si) substrate was used to place the solid samples. Iron (II, III) oxide (Fe₃O₄, product no. −637106 from Sigma-Aldrich) solid powder was used as a control to compare the Raman spectral behavior of the synthesized IO nanoparticles. Raman spectroscopy was carried out in a Renishaw In-Via Raman spectrometer equipped with a 488 nm laser and motorized stage. The spectra are collected at five percent laser power (19.47 micro-Watt) for a serial exposure time of 50 seconds per acquisition. Five successive acquisitions were carried out to improve the signal to noise ratio.

### Example 7. Preparation and Characterization of Chitosan-Coated Composite Ink

Chitosan-based composite ink formulations were prepared by vigorously mixing equal volumes of neutralized (pH of 6-7) chitosan solutions and different contrast nanoparticles (for example, dextran-coated iron oxide, dextran-coated copper oxide, carbon nanotube, graphene oxide etc.) for 24 hrs. The composite ink materials were further characterized for its hydrodynamic diameter and surface zeta potential using Dynamic Light Scattering (DLS) as illustrated in FIGS. 7 and 8 respectively. The characterization of dextran-coated iron oxide nanoparticle (DFeNP) shown in FIGS. 7 and 8 was performed using an aqueous dispersion at 25 mg/mL. ICP-OES: Fe estimation was 0.533±0.05 mg Fe per mg of DFeNP sample. The results indicate the presence of primarily Fe₃O₄ by composition (Qualitative verification performed by Raman Spectroscopy).

Particle size was monitored over the period of several weeks to identify any potential instability for long-term storage. Viscosity and magnetic moment were measured for additional assessment of the composite ink materials.

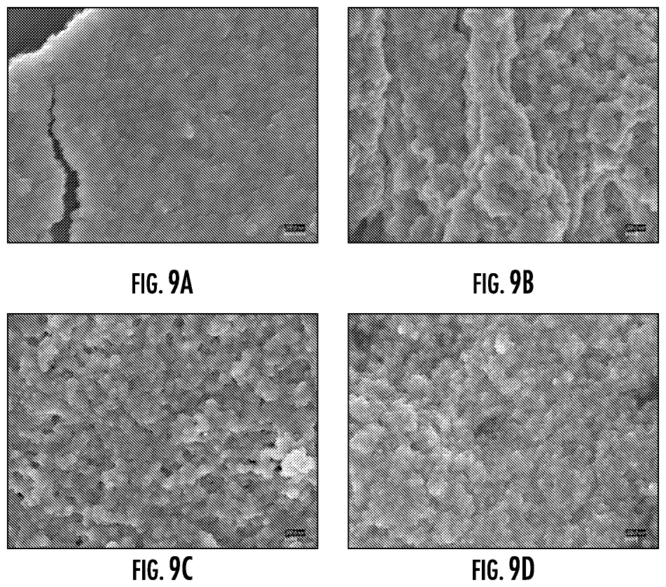

FIGS. 9A-9D show scanning electron microscopy (SEM) images of dextran-coated Iron Oxide (IO) nanoparticles (FIG. 9A); high molecular weight-chitosan coated IO-nanoparticles (hC-IO) (FIG. 9B), glutathione-conjugated chitosan coated IO-nanoparticles (gC-IO) (FIG. 9C), and quaternized-chitosan coated IO-nanoparticles (qC-IO) (FIG. 9D). Scale bars are 200 nm.

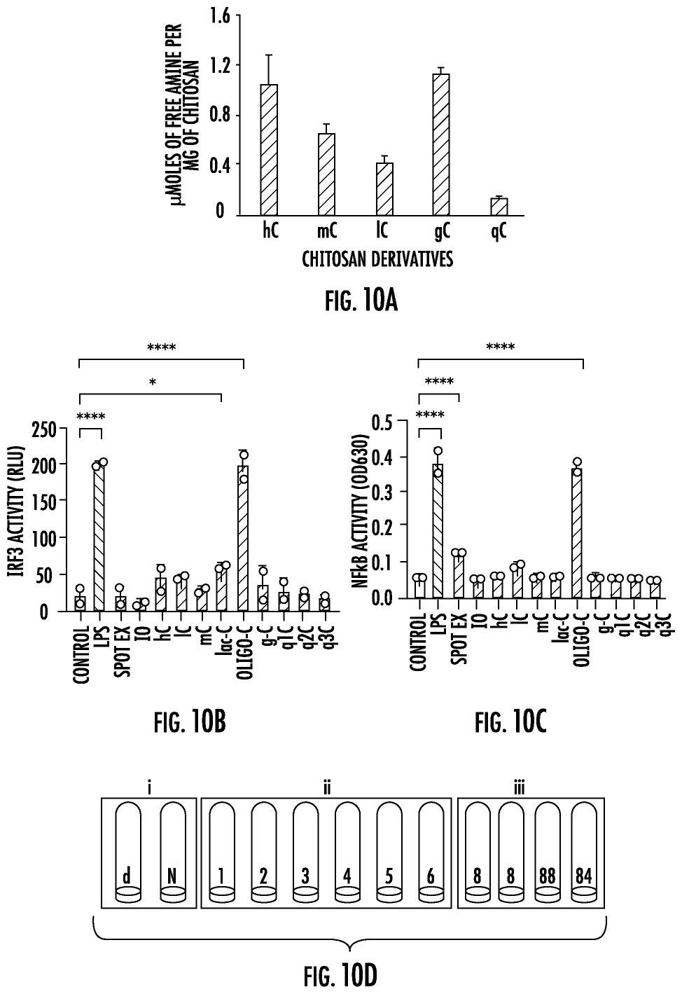

FIG. 10A shows a comparison of free amine present in different chitosan derivatives measured with Ninhydrin assay. FIGS. 10B and 10C show in vitro inflammation assay response of different tattoo-ink components such as IO nanoparticles and chitosan derivatives towards IRF3 (FIG. 10B) and NFKB (FIG. 10C) activation with J774-dual mouse-derived macrophage-based cells. ‘Control’ refers to culture media as negative control and ‘LPS’ refers to positive control. FIG. 10D depicts Representative images of gel clot assay to detect endotoxin present. (I) represents positive (P) and negative (N) controls of the assay with detection limit of 0.25 EU/mL. (II) represents no gel clot formation in different neutralized chitosan derivatives used (marked as 1-4) and different batches of synthesized IO nanoparticles (marked as A. B). (III) represents no gel clot formation in chitosan-based IO-composite tattoo-inks (marked as 1B-4B).

### Example 8. Ex Vivo Implantation of Composite Ink in Porcine Intestines

Fresh porcine intestines were collected, further dissected, and washed with saline. In particular, the meat processing plant from where we received the fresh porcine intestines for ex vivo studies) was West Valley Processing.

Then a portion of wet intestinal tissue was placed on an inverted weighing boat and known volume (40-70 μL) of composite ink was injected into the submucosal layer with an insulin syringe at ˜10° angle with tissue surface. The tattoo/mark was visualized immediately and captured using a cellphone camera. All the images were processed using a software (ImageJ) and further evaluated for measuring contrast and area of each spot created with the composite ink (tattoo). Contrast ability of our synthesized ink was found comparable with the commercially available Spot-Ex® ink and total area of the spot/mark created by the tattoo was much smaller/localized with our composite ink material, in comparison with commercial Spot-Ex® ink.

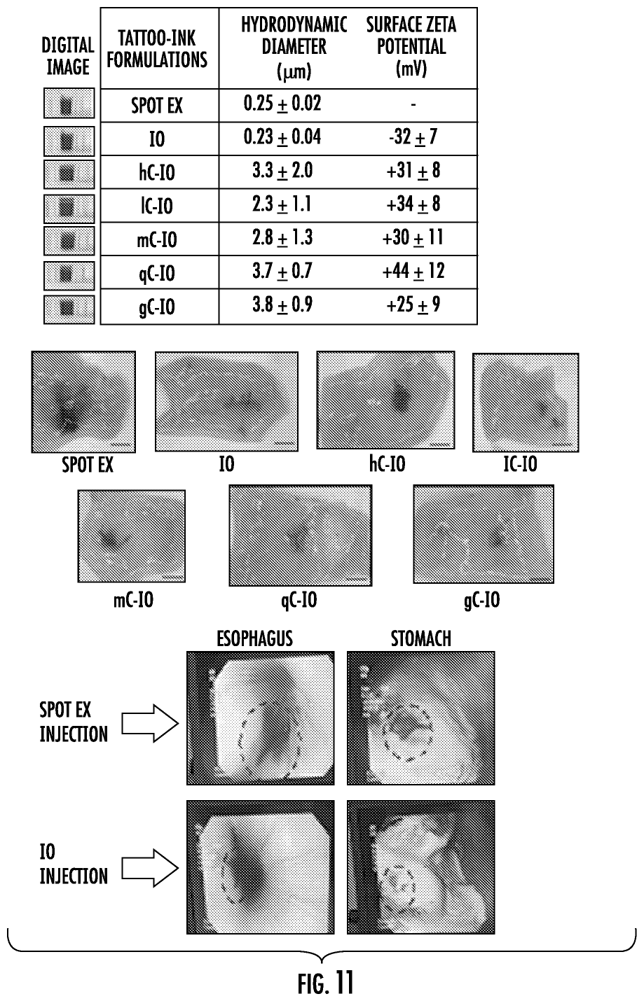

FIG. 11A presents physical appearance of different tattoo-inks taken in a cuvette and a table depicting hydrodynamic diameter & zeta potential measurements of different tattoo-inks using dynamic light scattering. FIG. 11B shows representative images of ex vivo tattoo performed with different tattoo-ink formulations using fresh porcine intestinal tissue. FIG. 11C shows images of preliminary trials (N=1) of endoscopic injections into esophagus and stomach of a just-sacrificed porcine with the commercial Spot® Ex ink and synthesized IO-nanoparticles.

### Example 9. In Vitro Inflammation Assay with Chitosan Derivatives

The results in Table 1 were obtained for in vitro inflammation assay with Chitosan derivatives. All samples were tested negative to 0.25 EU/ml on the same day with an LAL assay.

Derivatives are as defined herein. DCuNP is dextran coated copper nanoparticle, Traut's Reagent is 2-immunothiolane which can be complexed with chitosan. LPS is Lipopolysaccharide.

TABLE 1
In Vitro Inflammation Assays with Chitosan Derivatives.

| | NFkB | IRF3 |
|---|---|---|
| Control | 0.0605 | 20.5 |
| LPS | 0.379 | 200 |
| HMW-CH | 0.063 | 46 |
| MMW-CH | 0.0625 | 30 |
| LMW-CH | 0.093 | 47.5 |
| CH-Lactate | 0.064 | 62.5 |
| CH-Oligo | 0.365 | 197 |
| CH-Cys | 0.062 | 25.5 |
| CH-GSH | 0.06 | 35 |
| CH-Catechol | 0.0595 | 11.5 |
| CH-Traut's | 0.058 | 39.5 |
| CH-DDA1(40%) | 0.0585 | 6.5 |
| CH-DDA2(56%) | 0.0595 | 23 |
| CH-DDA3(69%) | 0.059 | 23 |
| Q-CHI | 0.0595 | 30.5 |
| Q-CH2 | 0.06 | 25 |
| Q-C53 | 0.0575 | 15.5 |
| DCuNP | 0.072 | 8 |
| DFeNP | 0.0585 | 10 |
| Spot-EX | 0.1295 | 20.5 |

### Example 10. Comparison of 3-Day In Vivo Study: Spot-Ex Versus DFeNP

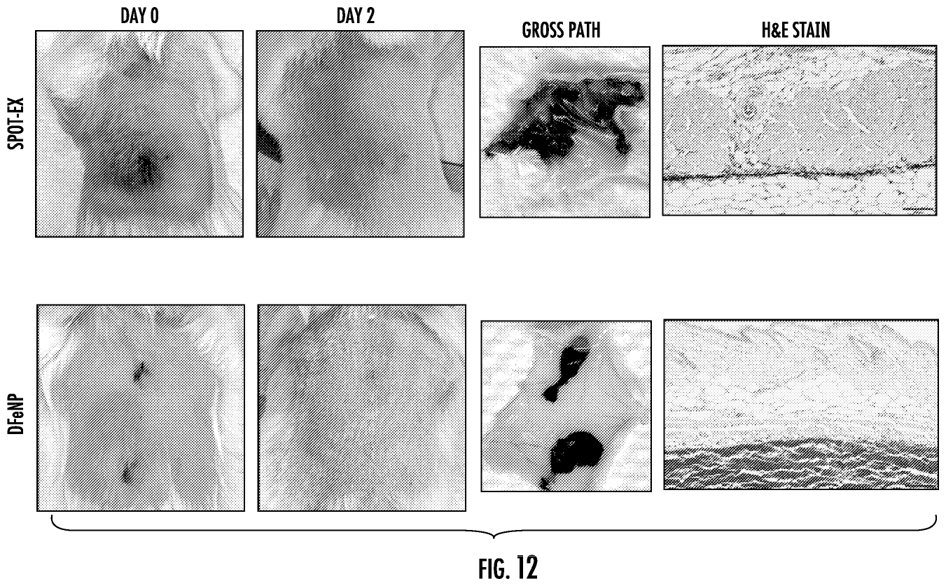

Two SubQ injections (50 μL/injection) with same ink were made on a mouse. As seen in FIG. 12, observation at two days shows the injections with Spot-EX in merged into one bigger spot. With the chitosan-based complexes disclosed herein, the two injections using DFENP remained as two spots.

### Example 11. Comparison of 28-Day In Vivo Mice Studies

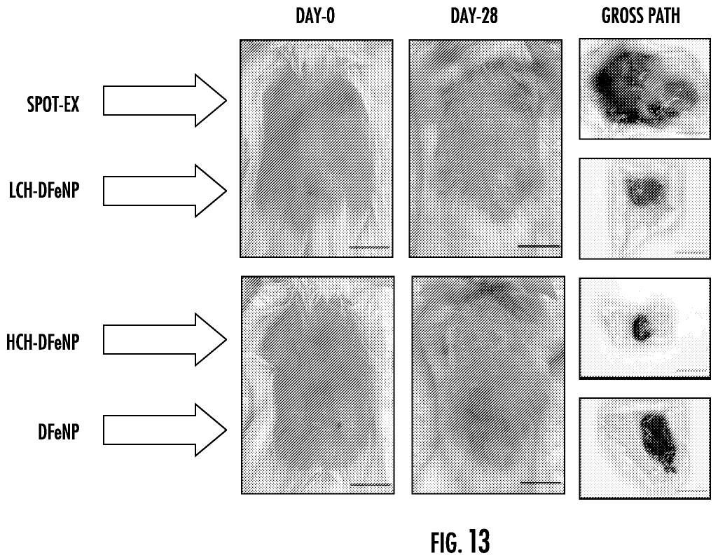

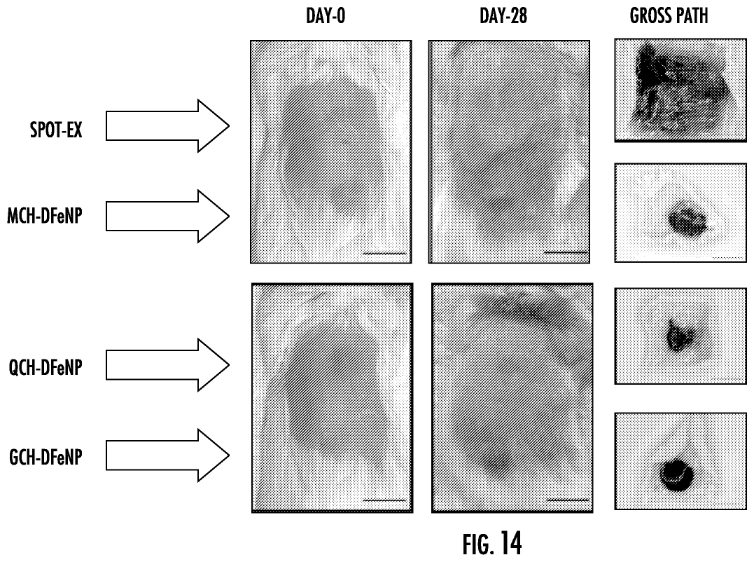

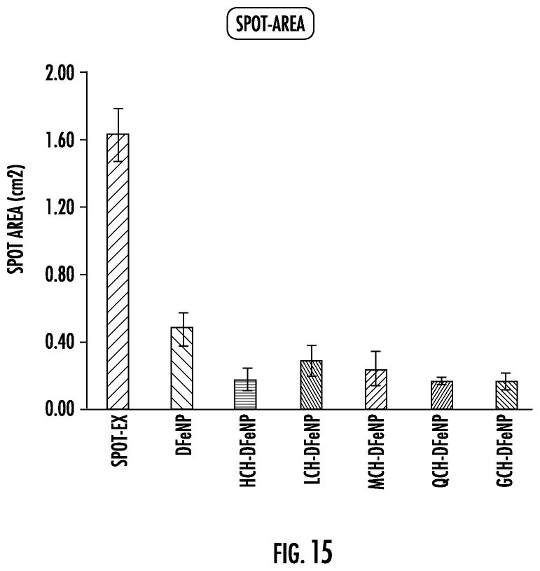

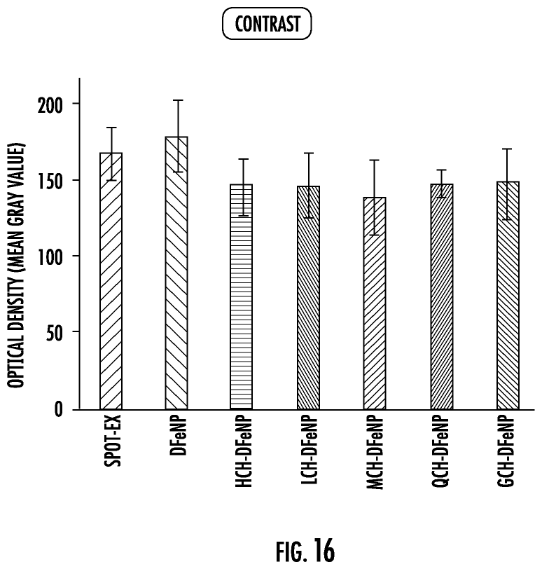

Two site injections as described above were utilized. FIG. 13 shows representative images at 28 days for an in vivo first cohort mice sturdy using Spot-Ex, LCH-DFeNP, HCH-DFeNP, and DFeNP. FIG. 14 presents representative 28 days for an in vivo second cohort mice study using Spot-EX, MCH-DFeNP, QCH-DFeNP, and GCH-DFeNP.

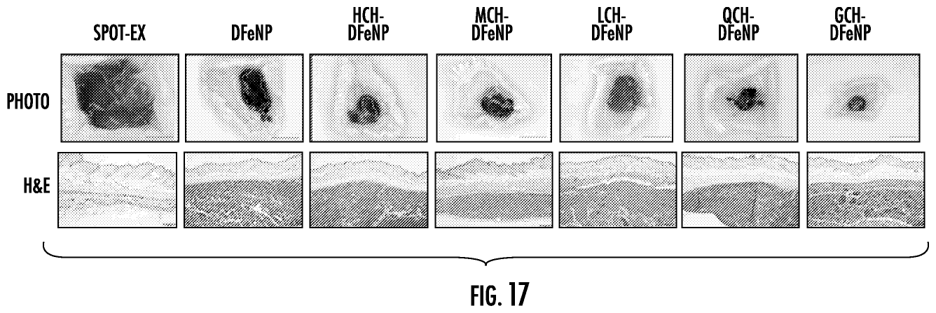

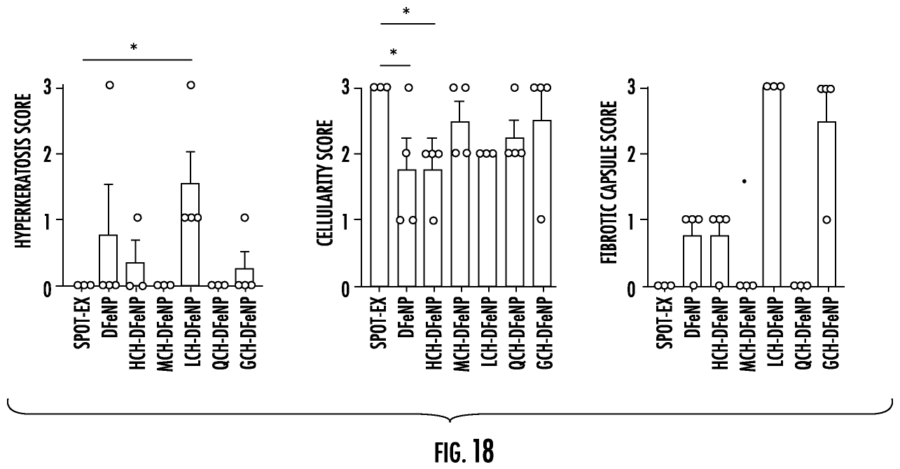

Graphs showing size of the spot area and the contrast are presented in FIGS. 15 and 16 respectively. The spot size was significantly larger for the Spot-Ex agent versus the other agents. There was no significant difference in contrast between the different dyes.

Images and hematoxylin-eosin stain (H&E) of the dye injection sites are shown in FIG. 17. Hyperkeratosis scores, cellularity scores, and fibrotic capsule scores for the tested dyes are presented in FIG. 18.

### Example 12. Ex Vivo Evaluation of Tattoo Ink Composites

Fresh porcine intestines were collected, further dissected, washed with saline and store in −20° C. for future use. The intestinal tissue used in this ex-vivo studies were collected from porcines (breed-yorkshire) of age between 1-2 years. Then a portion of wet large-intestinal tissue was placed on an inverted weighing boat and a bleb (with ˜1 mL saline solution) was created to expand the submucosal layer, followed by injections of known volume (50 μL) of composite tattoo-ink into the submucosal layer with a 28 G insulin syringe at ˜10° angle with luminal tissue surface. The tattoo/mark was visualized immediately and captured using a cellphone camera placing into an enclosed black box chamber to avoid any lighting artifacts and maintain uniform light condition. All the images were processed using a software (ImageJ) and further evaluated for measuring contrast and area of each spot created with the composite tattoo-ink.

Tattoo-ink samples were safely transported within an enclosed box to Mayo Clinic Animal research facility to perform endoscopic injections into esophagus and stomach of a porcine (which was sacrificed within an hour before the endoscopic injections were performed). 1 mL of saline was injected first to create a bleb in the submucous layer and followed by the injection of ˜1 mL of tattoo-ink solution in each case. Following the study ex vivo tissues were collected and fixed with formalin solution for further histopathological analysis.

### Example 13. In Vitro Immune Activation Study

J774-DUAL is derived from a mouse macrophage-like cell line (Snyderman et al., 1977) by simultaneous stable transfection with the NFKB-inducible SEAP gene and the ISRE-inducible Lucia gene. J774-DUAL murine-derived macrophages (from ATCC, Rockville Pike, MD, USA) were cultured in 5% CO₂ atmosphere in DMEM supplemented with glutamax-1 containing 10% heat-inactivated FBS, 100 U·mL⁻¹ penicillin, 100 mg mL⁻¹ streptomycin and 250 ng mL⁻¹ amphotericin B (all from Invitrogen). For experiments, cells were seeded on 96-well plates at a density of 50,000 cells per well and cell monolayers were grown for 24 hours before the immune activation experiments were started. All the test samples were initially diluted by 10-fold from its original stock solution and then 10 μL of diluted test samples were mixed with 190 μL of cell culture media to attain another 20-fold dilution, which was directly used to treat the cells. Original cell culture media was used as a negative control and lipopolysaccharide (LPS) was used as a positive control in this assay. Following 24 hours of the treatment, Quant-Blue and Quant-Luc assays were performed using the supernatant solution by following the procedure supplied with the kit. NFKB activity was determined by measuring secreted alkaline phosphatase (SEAP) using a colorimetric assay and to determine IRF3 activity by measuring secreted luciferase using a luminescence-based assay.

ToxinSensor™ Gel Clot Endotoxin Assay kit from GenScript was used and the procedures supplied with the kit; was followed to determine the level of endotoxin present in the samples with a detection limit of 0.25 EU/mL. LPS was used as a positive control and endotoxin-free water or original cell culture media was used as a negative control.

### Example 14. In Vivo Studies in Mice

All animal care and experimental studies were carried out in compliance with protocols approved by the Institutional Animal Care and Use Committee (IACUC) at Arizona State University. Studies were performed with equal numbers of male and female immunocompetent Balb/c mice aged between 8-12 weeks (weighing 22-25 g), purchased from Charles River Laboratories, Inc. (Wilmington, MA) and were housed in groups at the ASU animal care facility. Mice were anesthetized by isoflurane (1-3%) and a region measuring approximately 1 inch wide by 1.5 inches long (along the spine) were shaved on the back centered between shoulders (intrascapular) using an electric shaver. The region was further sterilized by sequential washes with chlorhexidine gluconate and 70% ethanol in two rounds. Two spots were marked with a pre-printed pattern using washable ink spaced approximately 1 cm apart along the spine. The two sites were subcutaneously injected with 50 μL of synthesized biomaterial-based tattoo-ink or 50 μL commercial Spot® Ex dye as a standard (concentration is proprietary, will use as-is), or 50 μL of 0.9% normal saline as a control. All tattoo-ink materials were synthesized aseptically and sterilized by UV irradiation overnight prior to the day of the procedure. All synthesized tattoo-ink materials were confirmed negative for detectable endotoxin content (with detection limit of 0.25 EU/mL) using above-mentioned gel-clot assay prior to use for in vivo studies. Two subcutaneous injections were performed to evaluate the degree of diffusion within the subcutaneous space and the localization of immune reaction to our synthesized tattoo-ink materials. The markings were gently washed off with a damp sterile lab wipe, photographs of the spots were taken, and the mice were transferred to a cage to recover. Mice were monitored until fully awake and active before returning to the colony.

Mice were monitored daily until the follow-up date (day-3 for short-term studies and day-28 for long-term studies), including weekends. Photographs were collected to evaluate potential contrast-power and monitor any sign of dermal reactions. At the day of follow-up, mice were euthanized by CO₂ asphyxiation and cervical dislocation. Full-thickness skin samples were collected and placed in formalin for fixation and histologic analysis. The tattoo/mark was visualized immediately and captured using a cellphone camera placing into an enclosed black box chamber to avoid any lighting artifacts and maintain uniform light condition. All the images were processed using a software (ImageJ) and further evaluated for measuring contrast and area of each spot created with the composite tattoo-ink.

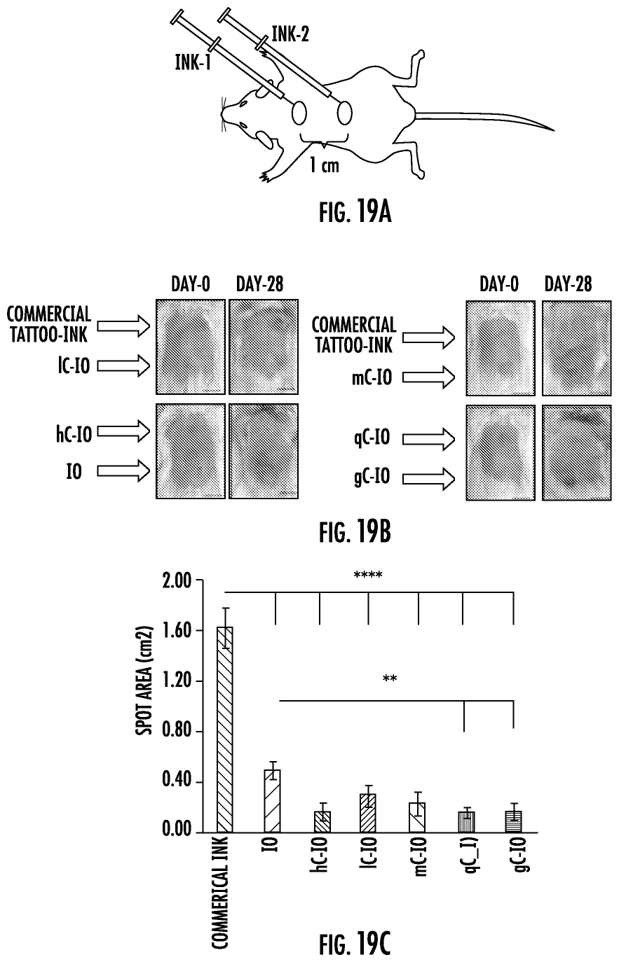

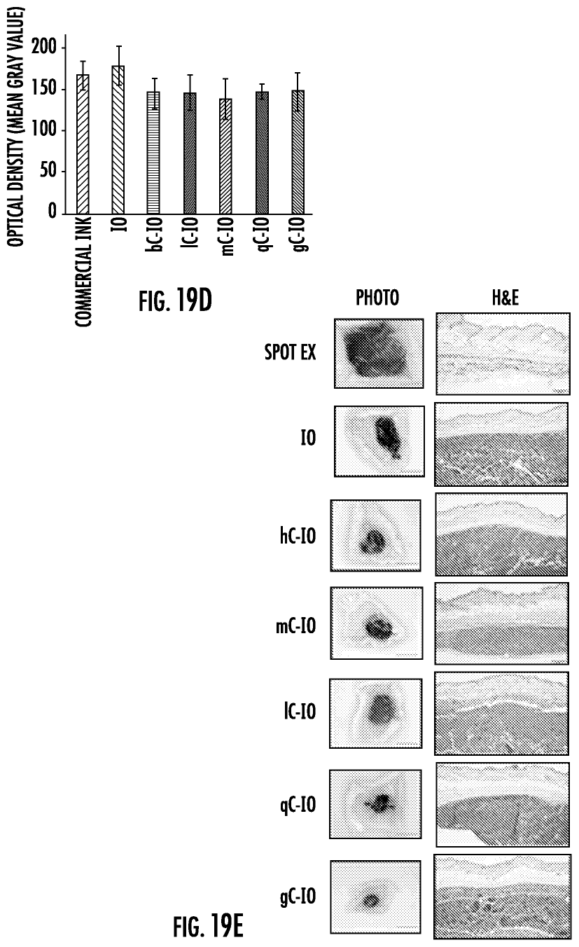

FIG. 19A shows schematics of randomized subcutaneous tattoo-ink injections during 28-day in vivo studies in live Balb/c mice. FIG. 19B shows images of tattoo-ink implanted mice at the start (day-0) and end (day-28) of the in vivo studies. FIGS. 19C and 19D depicts a comparison of spot-area (FIG. 19C) and contrast quantification (FIG. 19D) between commercial ink (Spot® Ex) and the disclosed tattoo-ink formulations. FIG. 19E shows representative gross pathology and H&E-stained images of skin tissues isolated following sacrificing the mice at end of the study (day 28). Subsequent histopathological analysis was performed to identify lead candidates for the future trials based on its minimal inflammation response determined by low/no hyperkeratosis score, low cellularity, and low capsule formation in vivo.

The dyes of the instant disclosure showed low inflammation in vitro, good contrast, good spot-area localization, low cellularity, low capsule formation, and low/no hyperkeratosis in vivo. As such, the instantly disclosed dyes show improvements and advantages to currently used dyes.

As required, detailed embodiments of the present invention are disclosed herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the invention to be better understood. However, they are given merely by way of guidance and do not imply any limitation.

The present invention may be understood more readily by reference to the following detailed description taken in connection with the accompanying figures and examples, which form a part of this disclosure. It is to be understood that this invention is not limited to the specific materials, devices, methods, applications, conditions, or parameters described and/or shown herein, and that the terminology used herein is for the purpose of describing particular embodiments by way of example only and is not intended to be limiting of the claimed invention. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention, which are, for clarity, described herein in the context of separate embodiments, may also be provided in combination in a single embodiment. Conversely, various features of the invention that are, for brevity, described in the context of a single embodiment, may also be provided separately or in any sub-combination. Further reference to values stated in ranges includes each and every value and combination of values within that range.

The following definitions are intended to assist in understanding the present invention.

As used herein, the singular forms “a,” “an,” and “the” include the plural, and reference to a particular numerical value includes at least that particular value, unless the context clearly dictates otherwise. Thus, for example, reference to “a step” includes reference to one or more of such steps.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

1. A composition of biomaterial-based composite ink comprising:
at least one contrast agent selected from the group consisting of: iron oxide, copper oxide, carbon nanotube, and graphene oxide; and
at least one polysaccharide-based biomaterial selected from chitosan or its derivative.

2. The composition of claim 1, wherein the at least one polysaccharide-based biomaterial is selected from the group consisting of cysteine-modified chitosan, glutathione-modified chitosan, and catechol-modified chitosan.

3. The composition of claim 1, wherein the at least one contrast agent is a nanoparticle.

4. (canceled)

5. The composition of claim 1, wherein the at least one contrast agent is coated.

6. The composition of claim 4, wherein the at least one contrast agent is coated with dextran.

7. The composition of claim 1, wherein the composite ink comprises dextran-coated iron oxide nanoparticles or dextrose coated iron nanoparticles encapsulated with quaternized chitosan, medium molecular weight chitosan, or high molecular weight chitosan.

8. The composition of claim 7, wherein the dextran-coated iron oxide nanoparticle comprises 0.533+/−0.05 mg iron per mg of DFeNP.

9. The composition of claim 1, wherein the contrast agent has an about 60 to about 500 nm hydrodynamic particle diameter.

10. A method of endoscopic tattooing comprising injecting to tissue a composition comprising biomaterial-based composite ink comprising:
at least one contrast agent selected from the group consisting of: iron oxide, copper oxide, carbon nanotube, and graphene oxide; and
at least one polysaccharide-based biomaterial selected from chitosan or its derivative, wherein the at least one contrast agent is encapsulated with the at least one polysaccharide-based biomaterial.

11. The method of claim 10, wherein the at least one contrast agent is a nanoparticle.

12. (canceled)

13. The method of claim 10, wherein the at least one contrast agent is coated.

14. The method of claim 13, wherein the at least one contrast agent is coated with dextran.

15. The method of claim 10, wherein the at least one polysaccharide-based biomaterial is selected from the group consisting of cysteine-modified chitosan, glutathione-modified chitosan, and catechol-modified chitosan.

16. The method of claim 10, wherein the at least one polysaccharide-based biomaterial is quaternized chitosan, medium molecular weight chitosan, or high molecular weight chitosan.

17. A method of synthesizing a composition of biomaterial-based composite ink comprising mixing:
at least one contrast agent selected from the group consisting of: iron oxide, copper oxide, carbon nanotube, and graphene oxide; and
at least one polysaccharide-based biomaterial selected from chitosan or its derivative, wherein the at least one contrast agent is encapsulated with the at least one polysaccharide-based biomaterial.

18. The method of claim 17, wherein the at least one contrast agent is a nanoparticle.

19. The method of claim 17, wherein the at least one contrast agent is coated.

20. The method of claim 19, wherein the at least one contrast agent is coated with dextran.

21. The method of claim 17, wherein the at least one polysaccharide-based biomaterial is selected from the group consisting of cysteine-modified chitosan, glutathione-modified chitosan, and catechol-modified chitosan.

22. The method of claim 17, wherein the at least one polysaccharide-based biomaterial is quaternized chitosan, medium molecular weight chitosan, or high molecular weight chitosan.
